(19) United States
(12) Patent Application Publication
(10) Pub. No.: US 2004/0097464 A1
(43) Pub. Date: May 20, 2004

(54) Mixture of non-sulfated fucose-based oligosaccharides, a cosmetic or pharmaceutical composition comprising said mixture and its use in cosmetics or pharmacy

(76) Inventors: Ladislas Robert, Santeny (FR); Alexandre Michel Robert, Santeny (FR); Catherine Sylvie Robert, Paris (FR); Jean-Luc Gesztesi, Sao Paulo (BR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/363,462

(22) PCT Filed: Sep. 11, 2001
(86) PCT No.: PCT/BR01/00114

(30) Foreign Application Priority Data
Sep. 11, 2000 (FR) 00/11545
Mar. 13, 2001 (BR) PI 0100955-9

(51) Int. Cl. A61K 31/715; C08B 37/00; A61K 39/108

(52) U.S. Cl. 514/54; 536/123; 424/259.1

(57) ABSTRACT

The present invention relates to a mixture of non-sulfated fucose-based oligosaccharides, characterized in that it comprises oligosaccharides of less than 13 saccharide units, comprising at least one fucose unit in a non-reducing end position, and in that at least it is capable of being obtained by means of a process that comprises at least one step of degradation of a polysaccharide from a microorganism of the gender Klebsiella pneumoiae subsp. pneumoniae. This new mixture presents significant activities on different components of the skin, providing a clearly visible anti-aging result, specially by an excellent thickening of the skin.

The present invention relates to a new mixture of non-sulfated fucose-based oligosaccharides and to its use mainly in products of topical application, for which an activity on the epithelial or conjunctive tissue is sought, in particular in products against aging, such as pharmaceutical and veterinary products and, more particularly, in cosmetic products.

While aging the skin becomes thin by approximately 6% on an average every ten years (Skin thickness changes in normal aging skin, Branchet et al, Gerontology, 1990, 36: 28-35). The underlying mechanisms of aging are intrinsic and extrinsic. The intrinsic mechanisms comprise a decrease in cellular proliferation and an important loss of the cutaneous extramolecular matrix (CEM). This loss is mostly the result of the drop in the synthesis of the CEM and of the increasing synthesis of degradation enzymes that can attack the skin matrix, with the aging (D. L. ROBERT: aging, CNRS, Belin, 1994; Docteur L. ROBERT: aging, facts and theories, DOMINOS, Flammarion, 1995). The responsible proteases are essentially the matrix metalloproteases (MMP), several of which various (such as serines-proteases) may degrade most of the constituents of the skin matrix and, in particular, the elastic fibers.

In the last few years, numerous researches have been made with the objective of obtaining active compounds against certain effects of skin aging. One first objective is to render this process slower. Another objective is to obtain a result of thickening the skin, mainly the dernis.

The saccharides and polysaccharides are well-known substances in cosmetics, mainly for their hydrating properties. This is the case, for instance, of the monosaccharide fucose and of the polysaccharides that contain it.

Fucose is a deoxy-hexose close to galactose, of which it has the steric conformation. However, the structure of fucose essentially differs from that of galactose in that the C6 atom bears a methyl group (—CH₃) and not a primary alcohol group (—CH₂OH). Indeed, this methyl group imparts to the molecule of fucose an interesting partial hydrophobic nature, compensated for by the hydroxyl groups on which other atoms on the four carbon atoms present.

Fucose appears early during the phylogenesis, the polysaccharides of certain algae and of fungi that contain them in relatively important quantities, either alone or in combination with other chemical compounds. It may equally appear in sulfated form like the fucanes. On the other hand, fucose is widespread in the animal kingdom and certain bacteria also synthesize it.

However, in spite of the considerable number of papers on fucose and the polysaccharides that contain it, the results achieved so far have almost not been satisfactory for application on an industrial scale, mainly in a cosmetic product.

In particular, in addition to the poor cost/effectiveness relationship of the monosaccharide fucose, the known polysaccharides containing fucose do not present a significant activity, which enables one to fight against the skin aging, as set forth above.

Thus, for instance, fucanes are sulfated polymers with high molecular weight (>20 kD). WO 99/32099 (IFREMER) describes new uses of fucanes within the scope of repair of injuries of the conjunctive tissue. However, fucanes, due to their size and load, cannot interact effectively with all the cellular layers of the skin. In addition, fucanes can, on the contrary, activate the MMP-2, a redhibitory property for the cosmetic treatment of the normal aging of the skin.

FR-2 750 863 (L'OREAL) describes the use of a polyholoside, mainly a polysaccharide comprising fucose to favor scaling, that is to say, to favor the elimination of the “fly” cells located on the surface of the comeous layer of the epidermis (page 3, lines 6 and 7) and/or to stimulate the renewal of the epidermis, that is, to bring about forced elimination of the corneous layer that accelerates the renewal (page 1, lines 13-23), these effects bringing an assimilated result according to FR-2 750 863 to a result against skin aging. However, the polysaccharide as described in FR-2 750 863, for its molecular mass and its structure, does not allow one to integrate in a satisfactory way with the different constituents of the skin.

Then, it has been found, in an entirely surprising and unexpected way, that a new mixture of oligosaccharides containing fucose, which can be obtained by a specific treatment of the selected microorganism, presents significant activities on different components of the skin, bringing a real anti-aging result, clearly visible, mainly by an excellent thickening of the skin.

Thus, the present invention has the objective of providing a mixture of non-sulfated fucose-based oligosaccharides, characterized in that it comprises oligosaccharides of less then 13 saccharide units, comprising at least one fucose unit in a non-reducing end position, and in that it can be obtained by means of a process comprising at least one step of degradation of a polysaccharide from a microorganism of the gender Klebsiella pneumoniae subsp. pneumoniae.

By “non-sulfated fucose-based oligosaccharide” according to the invention, one understands, in accordance with the general knowledge of a person skilled in the art, an oligosaccharide containing at least one fucose saccharide unit and not bearing a sulfate group —O(SO₃)⁻. Fucanes are, in particular, excluded from this definition.

By “oligosaccharide comprising at least one fucose unit in a non-reducing end position”, according to the invention, one understands, in accordance with the general knowledge of a person skilled in the art, an oligosaccharide containing at least one fucose saccharide unit in an end position of the oligosaccharide chain, this fucose unit being linked to the next saccharide unit of the rest of the oligosaccharide by an acetal-type linkage.

The numbers of saccharide units may be measured with the aid of techniques known to a person skilled in the art, in particular, by applying the chromatography HPLC technique as described in the following examples.

Preferably, the mixture of oligosaccharides of the invention comprises, with respect to the total weight of the mixture, at least 15% by weight and, more particularly and preferably, from 20 to 50% by weight of oligosaccharides of less than 13 saccharide units, comprising at least one fucose unit in a non-reducing end position.

More particularly, the mixture of oligosaccharides, according to the invention, is characterized in that it further comprises, with respect to the total weight of the mixture, from 25 to 45% by weight of oligosaccharides having from 13 to 24 saccharide units, comprising at least one fucose unit in a non-reducing end position.

Still more particularly, the mixture of oligosaccharides of the invention is characterized in that it further comprises, with respect to the total weight of the mixture, from 15 to 35% by weight of oligosaccharides of more than 54 saccharide units, comprising at least one fucose unit in a non-reducing end position.

The mixture of oligosaccharide is capable of being achieved by means of a process that comprises at least one step of degradation of a polysaccharide from a microorganism of the gender Klebsiella pneumoniae subsp. pneumoniae, the oligosaccharides preferably comprising, at least in part, the motif of fucose-galactose galacturonic acid.

In particular, the mixture of oligosaccharides, according to the invention, is capable of being achieved by the process the comprises the steps of:

a) causing the microorganism of the gender Klebsiella pneumoniae subsp. pneumoniae to grow in an aqueous nutritive medium by aerobic fermentation of an assimilable source of glucide;

b) recovering the polysaccharide formed from the fermentation must;

c) subjecting the formed polysaccharide to a moderate hydrolysis;

d) subjecting the hydrolysis product of the step c) to an enzymatic hydrolysis; and

e) deactivating the enzyme after recovering the mixture of oligosaccharides thus formed.

More particularly, these steps a)-e) may be described in the following way:

Step a)

One preferably uses the microorganism Klebsiella pneumoniae subsp. pneumoniae, which is the microorganism deposited in the Collection Nationale de Cultures de Microorganismes under the number I-1507, or a mutant thereof. On the other hand, this microorganism is described in detail in application WO 96/23057.

The aqueous nutritive medium may be any aqueous medium known to a person skilled in the art that contains sources of carbon, nitrogen and mineral salts, such as those described in application WO 96/23057.

The fermentation may be conducted at temperatures on the order of from 25 to 35° C., with a pH of about 6.0 to 7.5, under conditions of aeration and stirring, for periods of from 2 to 4 days.

The fermentation may be made in a classic fermenter, inoculating the previously sterilized nutritive medium, for example, by heating up to a temperature of about 120° C. or by sterilizing filtration.

Step b)

At the end of the period of fermentation, the fermentation must is recovered, and a fucose-rich polysaccharide is isolated from it in the following way:

The fermentation must is subjected to a heat treatment at a temperature specially ranging from about 100 to about 130° C., preferably from about 115 to about 125° C., for about 30 minutes to about 2 hours and, preferably, from about 40 minutes to about 1 hour and with a pH specially ranging from 2 to about 5.5 and, preferably, from about 3 to about 5.5.

The product of the heat treatment is filtered according to classic means such as a press filter with plates.

In this way, one obtains a limpid, viscous polysaccharide, free from any cell.

Then a precipitation is carried out in an alcohol solvent, preferably an alcohol solvent chosen from ethanol, isopropanol and mixtures thereof. In particular, one uses from about 1 to about 3.0 volumes of solvent to 1 volume of polysaccharide and, preferably, from about 1.3 to about 2.0 volumes of solvent to 1 volume of polysaccharide.

Then, one carries out the drying under vacuum at a temperature specially ranging from about 20 to about 60° C. and, preferably, from about 30 to about 50° C., until a powder is obtained.

Steps c) and d): Hydrolysis of the Polysaccharide

This is an essential combination of steps. Indeed, one has found, in a surprising and unexpected way, that the combination of a step of moderate hydrolysis, preferably by irradiation with gamma rays and/or by protolysis, with an enzymatic hydrolysis step, enables one to obtain advantageously a sufficient global output of hydrolysis, close to that of a classic hydrolysis, such as an acidic hydrolysis, but with the advantage of specific cuts of an enzymatic hydrolysis. In particular, an acidic classical hydrolysis does not enable one to obtain a mixture of specific oligosaccharides, according to the invention, as it leads to the obtention of statistic, redhibitory cuts, as to the random nature. In addition, the compounds resulting from a classic acidic hydrolysis prove to be biologically inactive.

Step c): Moderate Hydrolysis of the Polysaccharide

The moderate hydrolysis is carried out by a treatment with gamma rays, a protolysis treatment or by these two successive treatments. Preferably, one successively carries out a treatment with gamma rays and then a protolysis treatment.

The treatment with gamma rays proved to cause a sensible drop in viscosity by a limited degradation, attributable to the action of free radicals. It may be carried out with irradiation means known to those skilled in the art.

This treatment by gamma rays, which are very penetrating rays, presents, in addition, the advantage of sterilizing the polisaccharide, killing the germs present, which could induce inflammation or even cause granuloma. In this way, one prevents a bacterial attack, without having to add to the medium any antiseptic products that could interfere in an undesirable way with the biologic activities of the end product.

The polysaccharide powder obtained in step b), possibly irradiated with gamma rays, may therefore, equally, be subjected to a protolysis treatment. For this purpose, it is placed in an aqueous solution, specially at the proportion of from 1 to 20% by weight and, preferably, from 2 to 10% by weight, with respect to the total weight of the aqueous solution.

The aqueous solution is subjected to a heat treatment, that is to say, a heating up to a temperature specially ranging from about 75 to about 120° C. and, preferably, from about 90 to about 100° C., for a period of time ranging from 1 to 6 hours, in the presence of a proton-generating resin, such as those commercialized and well known to a person skilled in the art, that is to say, a resin generating protons that bring about a cut of the glucosidic linkages with fixation of a water molecule.

Step d): Enzymatic Hydrolysis

One introduces an acidic buffer such as a citric acidic buffer (4.15 g/kg)-disodium hydrogenophosphate (about 10.75 g/kg) in the hydrolysate obtained in step b). One regulates the temperature of the solution specially to a temperature ranging from about 25 to about 45° C. and, preferably from about 30 to about 40° C.

One introduces an enzymatic preparation comprising at least one endofucosidase, preferably Fermizyme HCP such as commercialized by Gist Brocades, according to contents specially from about 2 to about 20% by weight and, preferably, from about 5 to about 15% by weight, with respect to the initial weight of polysaccharide powder utilized.

The thus obtained mixture is maintained under stirring for a period of time ranging from about 8 to about 24 hours and, preferably, from about 10 to about 20 hours, at a temperature specially ranging from about 25 to about 45° C. and, preferably, from about 30 to about 400° C., the pH being regulated at 6 by the presence of the buffer mixture.

Step e)

The hydrolysis product obtained after the step d) is filtered according to classical means such as a press filter with plates.

The collected solution is then heat-treated at a temperature specially ranging from about 75 to about 120° C. and, preferably, from about 90 to about 105° C., for a period of time specially ranging from about 10 to about 45 minutes and, preferably, from about 20 to about 35 minutes, in order to deactivate the enzyme and, more particularly, the fucosidase activity of this specific enzyme.

One let it cool down to a temperature specially ranging from about 20 to about 40° C.

While it is cooling, preservatives may by added to the solution.

One then filters the whole under sterile conditions, and then the packaging is carried out.

The thus obtained mixture of oligosaccharides according to the invention may be characterized with the aid of techniques well known to those skilled in the art, specially HPLC, chromatography on the thin layer and other methods and chemical dosages.

In this way, one can find out that the oligosaccharides of the mixture, according to the invention, are such that fucose is mainly at the end of the chain in a non-reducing end position.

The mixture of oligosaccharides is particularly suitable as an active mixture in a cosmetic composition that is specially anti-aging or in a pharmaceutical composition that is specially dermatological (topical application). In particular, the biologic effects found in this mixture of oligosaccharides are very comparable and even superior to those of the monosaccharide fucose.

Thus, the present invention equally has the objective of providing a cosmetic or pharmaceutical composition characterized by comprising, as a cosmetically or pharmaceutically active agent, at least one mixture of oligosaccharide such as described above and at least one cosmetically or pharmaceutically acceptable excipient.

When the composition is a pharmaceutical one, it is preferably a dermatological composition, for topical application, thus having at least one pharmaceutical excipient suitable for this dermatological application.

The cosmetically or pharmaceutically acceptable excipient may be any one from those known to a person skilled in the art for the purpose of obtaining a composition according to the invention in the form of a cream, a lotion, a gel, a salve, etc., possibly in the form of an emulsion, having, in addition, other components known to a person skilled in the art, to improve, modify or stabilize the composition from a cosmetic or pharmaceutical point of view.

The expression “pharmaceutically acceptable excipient” embraces excipients adapted for a veterinary use of the composition, according to the invention.

The composition according to the invention may, in particular, contain other additives and, as an aid to the formulation, such as antioxidant agents for fighting free radicals. One can cite specially pure vitamin E or dialpha-tocopherol and its derivatives, and 2,6-di-tert-butyl-p-cresol (BHT).

Advantageously, the composition according to the invention may further comprise, in particular, at least one additive chosen from the group consisting of the agents structuring the skin (such as squalane and sphingolipides), the moistening agents (such as glycerin and hydroxy prosilan C), the emollients (such as butylene glycol and cetyl lactate), the silicones (such as cyclomethicone), the sun protection agents (such as Parsol 1789 and Eusolex 6300), the emulsifiers (specially Carbopol 1342 associated to triethanolamine and soybean lecithin), the thickeners (notably xanthan gum), the scavengers (specially EDTA), the antioxidants (such as BHT described above), the fragrances, the preservatives, water and mixtures thereof.

Of course, the operational conditions for preparing the cosmetic or pharmaceutical composition according to the invention are part of the general knowledge of the art.

Preferably, the mixture of oligosaccharides is present according to a proportion ranging from about 0.001 to about 20% by weight and, more particularly and preferably, from about 0.1 to about 10% by weight with respect to the total weight of the cosmetic or pharmaceutical composition.

However, without wanting to be trapped by any theory, the advantageous activity of the mixture of oligosaccharides according to the invention, illustrated in the examples given hereinafter, would result, in particular, from its very great capacity of interacting with the cellular membranes via the hydrophobic functions of the highly available methyl groups of the fucose, and from its interaction with the mannose-fucose receptor of the skin cells, macrophages or Langerhans cells (Condaminet et al: Human epidermal Langerhans cells express the mannose-fucose binding receptor, Eur. J. Immuno. 1998, 28: 3541-3551). The set of these effects may be characterized as an effect stimulating cellular communication.

Thus, the present invention has also the objective of using the mixture of oligosaccharides, as described above, for preparing a composition intended to stimulate cellular communication between the cells of the skin.

On the other hand, the present invention has also the objective of using the mixture of oligosaccharides as described above, for prepanng a composition intended to stimulate the cellular proliferation of the keratinocytes of the skin.

In addition, the present invention has the objective of using the mixture of oligosaccharides, as described above, for preparing a composition intended to stimulate the cellular proliferation of the fibroblasts of the skin.

Finally, the present invention has the objective of using the mixture of oligosaccharides as described above, for preparing a composition intended to inhibit the synthesis of elastase-type proteases by the fibroblasts of the skin.

The fibroblasts of the human skin synthesize various proteases, some of which have an elastase-type activity. This is particularly true for MMP-2 and MMP-9 and for membrane MMP.

Hyaluronane added to the fibroblasts in low concentrations (1-2 mg/ml) increases the elastase-type activity significantly. Surprisingly and unexpectedly, we can see that the mixture of oligosaccharides according to the invention inhibits this stimulus. Since hyaluronane is constantly present in the pericellular environment, this inhibiting activity may be considered to enabling one to reduce the degradation of the skin matrix with the aging.

Thus, the present invention has equally the objective of using the mixture-of oligosaccharides, as described above, for preparing a composition intended to inhibit the superexpression of the proteases MMP-2 and MMP-9, induced by hyaluronane.

More particularly, the present invention has also the objective of using the mixture of oligosaccharides, as described above, for preparing a composition intended to inhibit the synthesis of the proteases MMP-2 and MMP-9 by the fibroblasts of the skin.

On the other hand, it has been shown recently that the activity of the MMP is critical in the case of sensitizing by contact (M. C. Lebre et al, Arcg. Dermatol. Res., 1999, 291: 447452). In the presence of MMP inhibitors, the Langerhans cells of the epidermis cannot migrate through the dermis, as is the case at the time of sensitizing by contact. In this way, inhibition of the activity of the MMP by the mixture of oligosaccharides, according to the invention, offers a real effect of decreasing the sensitivity of the skin to irritation.

Therefore, it is also an objective of the present invention to use the mixture of oligosaccharides, as described above, for preparing a composition intended to decrease the sensitivity of the skin to irritation.

The mixture of oligosaccharides according to the invention enables one to achieve effects of increasing the thickness of the epidermis and of the dermis. Statistically, these two effects are highly significant. When the thickness of the skin decreases with the aging, this trophic effect of the mixture of oligosacchariges according to the invention is extremely important in preventing skin aging.

More particularly, one can observe a reinforcement of the bundles of collagen of the dermis of a skin treated with the mixture of oligosaccharides according to the invention. Considering the importance of the role of these bundles of collagen in the steadiness of the skin, this is a greater anti-aging result.

Consequently, the present invention has also the objective of using the mixture of oligosaccharides, as described above, for preparing a composition intended to stimulate the deposit of collagen fibers on the dermis.

The mixture of oligosaccharides is used as described above, preferably according to a proportion ranging from about 0.001 to about 20% by weight and, more particularly and preferably, from about 0.1 to about 10% by weight, based on the total weight of the composition.

The thus prepared composition comprises, in addition, a cosmetically or pharmaceutically (specially dermatologically) acceptable excipient, such as those known to a person skilled in the art.

Finally, the present invention further has the objective of providing a method of cosmetic treatment of the skin, characterized by applying onto the skin a cosmetic composition comprising at least one mixture of oligosaccharides as described above. The cosmetic composition comprises, in addition, a cosmetically acceptable excipient, such as those known to a person skilled in the art.

The following examples are intended to illustrate the present invention and should not at all be interpreted as limiting its scope.

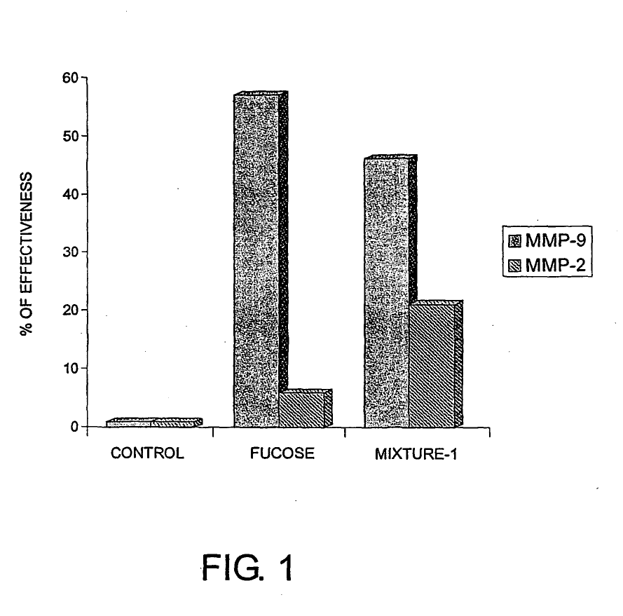

FIG. 1 is a histogram reporting the results presented in example 3.b.1, in terms of percentage of effectiveness for reducing the active form/inactive form relationship of the MMP-2 and MMP-9 secreted by explants of the skin.

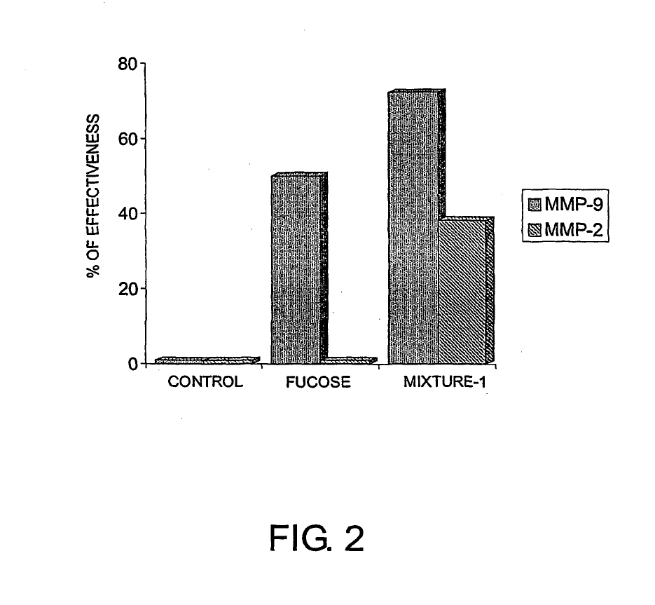

FIG. 2 is a histogram reporting the results presented in example 3.b.1, in terms of percentage of effectiveness for reducing the active form/inactive form relationship of the MMP2- and MMP-9 secreted by explants of skin stimulated by hyaluronane.

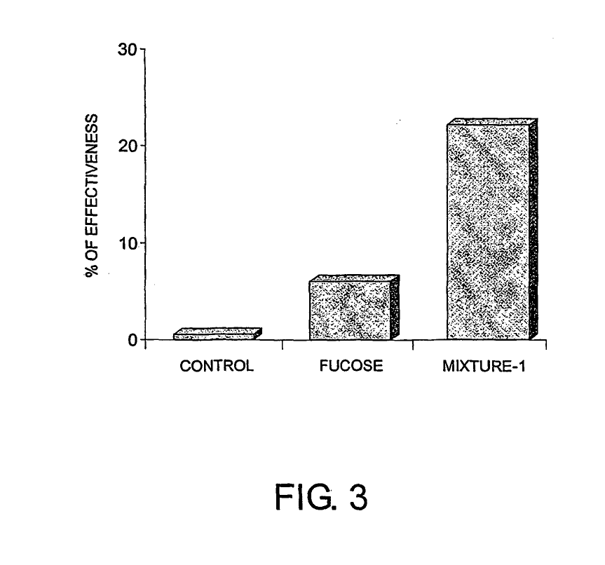

FIG. 3 is a histogram reporting the results represented in example 3.b.2, in terms of percentages of effectiveness for reducing the expression of the MMP-2 of explants of skin.

### EXAMPLE 1

### Preparation of a Mixture of Oligosaccharides According to the Invention

a) Fermentation

One uses the microorganism Klebsiella pneumoniae subsp. pneumoniae, which is the microorganism deposited in the Collection Nationale de Cultures de Microorganismes under number 1-1507. The nutritive medium and other fermentation conditions are as follows:

Preparation of the inoculums:

Culture Medium:

Neosorb® 70-07 (sorbitol contents: 70% M.S.; sold by ROQUETTE FRERES, Lille/France): 17.90 g/l (namely 12.5 gA of sorbitol)

Peptona Biokar 104003 (protein hydrolysate, sold by SOLABIABIOKAR, Pantin, France): −4.50 g/l

Yeast extract: 0.05 g/l

KH₂PO₄: 0.50 g/l

K₂HPO₄: 4.50 g/l

MgSO₄, 7H₂O: 0.20 g/l

Pluronic® PE 61000 (antifoaming agent), sold by BASF, D-6700

Ludwigshafen, Germany: 0.50 g/l

Placing in Solution Into Water

Culture Condition:

Sterilization at 121° C. for 30 minutes

Culture temperature: 30° C.

Inoculation rate: 5-10%

Aeration: 1 VVM

Non-regulated pH (pH approximately 7.00)

Culture Duration: 24 hours

Production Medium:

Culture Medium

Neosorb® 70-07: 54.00 g/l (namely 38 g/l of sorbitol)

Peptona Biokar®b 104003: 4.50 g/i

Yeast extract: 0.05 g/l

KH₂PO₄: 1.50 g/l

MgSO₄, 7H₂O: 0.20 g/l

Pluronic® PE 61000: 0.50 g/l

Placing in Solution Into Water

Culture Conditions (Chemap Fermenter Having Useful Volume of 350 Liters):

Sterilization at 120° C. for 45 minutes

Culture temperature: 30° C.

Inoculation rate: about 5%

Stirring: 300 rpm (Rushton-type stirrer)

Aeration: 1 VVM

pH regulated at 7.0 by NaOH 7N

Pressure: 100-200 mbars

Culture Duration: 60-65 hours

Average Values Achieved in Production:

Viscosity at the end of cycle: 40000 MPa.s (viscosimeters: Brookfield DV-II+model LV, movable SP 31, chamber SC4-34/13R, 30° C.)

Concentration of the Polysaccharide Produced in the Medium, Calculated in L-fucose:

2g/l (Dische and Shettles methods)

Sorbitol consumed: >35 g/l (in sorbitol)

NaOH at 20% by weight consumed: 15 liters/m³

Start of regulation of the pH: 16-17 hours, after inoculation of the fermenter

Final dry extract of the fermentation medium ˜20 g/l

b) Recovery of the Formed Polysaccharide

One subjects the fermentation must to a heat treatment at a temperature of 120° C. for 45 minutes and with a pH of 5.5. The product of the heat treatment is filtered with the help of a press filter with Seitz-type plates. In this way, one obtains a limpid, viscous polysaccharide, free from any cell. Then one carries out a precipitation in 1.5 volume of ethanol to 1 volume of polysaccharide. Then a drying is carried out under vacuum at a temperature of 25° C. until a powder is obtained. Considering the microorganism used, this polysaccharide is composed of repetitive units of trisaccharide fucose—galactose—galacturonic acid, and thus it presents the following structure:

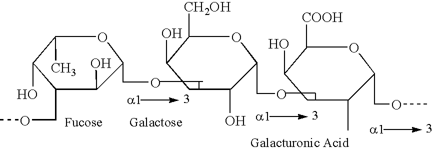

c) Moderate Hydrolysis of the Polysaccharide

The polysaccharide powder is placed in aqueous solution at the proportion of 5% by weight, based on the total weight of the aqueous solution. The aqueous solution is subjected to a heat treatment, that is to say, heating up to 100° C., for 3 hours, in the presence of a proton-generating resin.

d) Enzymatic Hydrolysis

One introduces the buffer mixture citric acid (4.15 g/kg)-disodium hydrogenphosphate (about 10.75 g/kg) into the hydrolysate. The temperature of the solution is regulated at 37° C. One introduces the enzymatic preparation Fermizyme HCP, as commercialized by Gist Brocades, according to contents of 20% by weight, based on the initial weight of polysaccharide used, that is to say, 0.05% by weight with respect to the total mass of the aqueous solution after placing the powder into water again, as described above for the moderate hydrolysis by protolysis.

The thus obtained mixture is kept under stirring for 15 hours at a temperature of 37° C., the pH being regulated at 6 by the presence of the buffer mixture.

e) Deactivation of the Enzyme and Recovery of the Mixture of Oligosaccharides

The product of hydrolysis is filtered with a press filter with Seitz-type plates. The solution collected is then heat-treated at 10° C., for 30 minutes, to deactivate the enzyme. One lets it cool at a temperature of 25° C. When it is cooling, the preservatives phenoxy ethanol (1% by weight) and phenonipe (0.3% by weight) are added to the solution. Then the whole is filtered in sterile conditions.

The thus obtained mixture of oligosaccharides is called “Mixture-1”.

### EXAMPLE 2

### Characterization HPLC of the Mixture-1

The fractionation of the Mixture-1 obtained in example 1 was carried out for the purpose of determining the proportion of oligosaccharides and polysaccharides in its composition.

a) Fractionation by Preparative Exclusion Chromatography on column “XK 50/60 Superdex 75 prepgrade”

One passes 50 ml of the Mixture-1 concentrated with 50 mg/ml, on a preparative column “XK 50/60 Superdex 75 prepgrade” (exclusion chromatography) and collects 95 fractions, then passed in HPLC.

TABLE 1
technical informations referring to the preparative column
XK 50/60 Superdex 75 prepgrade

| | |
|---|---|
| Packing | Superdex 75 prepgrade (34 μm) |
| Column size | Height: 50 cm |
| | Diameter: 60 mm |
| Column type | XK 50/60 |
| Usable interval of fractionation | 5 × 10² Da-3 × 10⁴ Da |
| Injected sample | 50 ml of the mixture-1 at concentration |
| | of 50 mg/ml (total: 2.50 g) |
| Elution Speed | 1 ml/minute |
| Number of collected fractions | 95 fractions of 12.5 ml each |
| Movable phase | PBS |

After fractionation the Mixture-1 on the column UXK 50/60 Superdex 75 prepgrade”, 95 fractions are collected, 45 of which contain osides.

b) Characterization of the fractions obtained by HPLC (exclusion chromatography), ultrahydrogel 120 and ultrahydrogel 250 columns

The objective of this second part of the study was to pass all the 95 fractions of the mixture-1 on a HPLC exclusion column (Ultrahydrogel 120 and 250 columns), in order to analyze the molecular weights and the concentration of the components of these fractions.

For this study one has worked with a Waters HPLC system, the description of which follows.

TABLE 2
technical characteristics of the HPLC chromatography
system used.

| | |
|---|---|
| Apparatus | HPLC Waters 600 |
| Columns | Ultrahydrogel 120 (pore size: |
| | 120 Å) and Ultrahydrogel 250 (pore |
| | size: 250 Å) from Waters. Size: 7.8 mm × 300 mm, |
| | containing the gel of |
| | hydroxylated polymethacrylate |
| Injected samples | 20 μl per automatic injector |
| Elution | 0.10 M NaNO₃ |
| Time of elution | 50 minutes/samples |
| Elution speed | 0.5 ml/minute |
| Detection | By measuring the refraction |
| | index with a Waters 410 refractometer |

TABLE 3
standards of molecular weights of polyethylene glycol
(Fluka) used for the HPLC exclusion chromatography

| Molecular weight | Time of elution (minutes) |
|---|---|
| 400 | 37.892 |
| 600 | 36.026 |
| 1000 | 33.940 |
| 2000 | 31.154 |
| 4000 | 28.896 |
| 6000 | 27.868 |
| 8000 | 26.946 |
| 12000 | 26.192 |
| 20000 | 25.308 |
| 35000 | 24.216 |

c) Results

The molecular weights of the components of the studied fractions were calculated, by using the following equation, obtained with the standards of molecular weights of Fluka, described in Table 3:

olecular weight of the components=55290000*10\hat{ }(−0.13942*x)

R²=0.982

X=time of elution (minutes)

The first fraction containing components of the Mixture-1 is fraction No. 44 and the last one is fraction No. 89, which means that the same component of the less elevated molecular weight is obtained after 89 fractions collected (after an elution of 89×12.5 ml=1112.5 ml). The fractions collected contain mono-, oligo- and polysaccharides of 184Da (mixture of monosaccharides) up to about 21 kDa. Therefore, this fraction contains polysaccharides formed by an average of 117 monosaccharide units or of 39 trisaccharide units.

Most of the fractions, with the exception of fractions No. 77, 78, 79, 81, 82, 83, 84, 85, and 86, contain a single saccharide peak (separation limited by the sensitivity of the separation method applied).

The approximate concentration of the different fractions may be determined by using an appraisal range of fucose standard at growing concentrations. This kind of “mono-compositional” appraisal range could be used thanks to the detection system (measure of the refraction index with a refractometer). According to these results, a solution of 1 μg/ml of fucose gives, on an average, a surface peak of 29409 (arbitrary units of the system). Knowing the surfaces of the peaks analyzed, it was possible to calculate their apparent concentrations.

The results achieved show that the Mixture-1 contains approximately 26% of small osides (up to 2 kDa, about 4 trisaccharide units), about 36% of oligosaccharides (up to 4 kDa, 8 trisaccharide units) and about 23% of polysaccharides of molecular weight higher than 10 kDa (18 trisaccharide units).

Considering the microorganism and the specific enzyme (endofucosidase) used for preparing the Mixture-1, it seems that the oligosaccharides of the mixture comprise a fucose unit in non-reducing end position.

### EXAMPLE 3

### Activity of the Fucose and of the Mixture-1 on the Activity of the Matrix Metalloproteases of Human Skin

Here one proposes to study the effect of the monosaccharide fucose and of the mixture of oligosaccharide “Mixture-1”, as prepared in Example 1 above, on the activity of the matrix metalloprotease (or MMP) of the fibroblasts in the quiescent state or stimulated by hyaluronane. Tests were carried out from cellular cultures of fibroblasts of human skin cultivated in monolayers, but also from culture of explants for studying the effect of the different treatments on a cellular environment closest to those of the skin. Indeed, the dermal fibroblasts are involved by an extracellular matrix abundant in vivo, a culture of explant is, therefore, a type of culture that reconstitutes the natural environment of the cells, thus enabling one to study the effect of the treatments on the activity of MMP of the fibroblasts in conditions close to the physiological conditions.

a) Method

a.1) Culture of Skin Explant

a.1.1) Activity MMP of the Non-Stimulated Skin Explants

Skin explants from a skin of the belly of a 54 years old woman were cultivated in a DMEM medium without phenol red (GIBCO) under stirring at 37° C. for 48 hours with different treatments. The samples were treated with a concentration of 10 μg/ml.

Recovery of the Samples:

Extracellular medium-1 ml of medium

Intracellular medium-explant ground with ultraturax in 1 ml of MMP buffer

MMP Buffer Composition:

0.1 M Tris-HCl, pH 7.5, 0.1 M NaCl, 10 mM CaCl₂, 1 mM zinc acetate

0.01% Brij 35, 0.01% NaN₃

Zymography

Gelatin, substrate of the MMP-s, was co-polymerized at final 1 mg/ml with a gel of polyacrylamide at 10%. The extracellular media from the cultures of skin explant were deposited on the top of the gel, the electrophoresis was carried out at 150 volts. The gels were then incubated in MMP buffer for 24 hours under stirring at 37° C. The gels were then colored with Coomassie blue, then uncolored with distilled water. The lise ranges corresponding to an MMP activity, appeared as white, while the background of the gel was blue. In function of the distance of migration of proteins, one could determine the molecular weights of the enzymes having digested the gelatin. The MMP activity was quantified by morphometric analysis with the aid of a Visiolab computer.

a.1.2) MMP Activity of the Skin Explants Stimulated with 12 mg/ml of Hyaluronane

The protocol of the studies was the same as that used for the study of the MMP activity of the non-stimulated skin explants, but with hyaluronane with a concentration of mg/ml and the samples of oligosaccharides with a concentration of 10 μg/ml, added in the culture medium.

a.2) Culture of Skin Fibroblasts

a2.1) MMP Activity of the Non-Stimulated Skin Fibroblasts

Skin fibroblast from a skin of the belly of a 45 years old woman were cultivated until the 7^{th} cellular passage in a DMEM Glutamax (GIBCO) medium containing 1% of antibiotics and of fungicide and 10% of fetal calf serum and were used for the experiment. The fibroblasts were inseminated on 6-well plates at a density of 5.10⁴ cells per well. When at the confluence, the culture medium was replaced by a DMEM medium, without phenol red containing 0.1% of BSA and containing the different substances to be treated at a concentration of 10 μg/ml. After 24 hours of culture at 37° C. (5% (v/v) CO₂, 95% (v/v) air) the samples were recovered.

Extracellular medium−1 ml of medium+2 rinsings with 0.25 ml of PBS

Intracellular medium−cellular carpet sonicated with 1 ml of MMP buffer

MMP Buffer composition 0.1 M Tris-HCl, pH 7.5, 0.1 M NaCl, 10 mM CaCl₂, 1 mM zinc acetate

0.01% Brij 35, 0.01% NaN₃

As in the case of the cultures of skin explants, the culture media of the fibroblasts were studied with zymography.

a.2.2) MMP Activity of the Fibroblasts of Skin Stimulated with 1 mg/ml of Hyaluronane

The protocol of the studies is the same as that used for the study of the MMP activity of the non-stimulated fibroblasts of skin, but with hyaluronane with a concentration of 1 mg/ml and the samples of oligosaccharides with a concentration of 10 μg/ml, added in the culture medium.

b) Results

b.1) MMP Activity of the Skin Explants

The MMP-s are enzymes that are secreted in the form of zymogens, inactive. Their activation is effected by proteolytic cleavage, the active form of the enzyme may then degrade the compounds of the extracellular matrix. Therefore, it is important to know not only the level of expression of the MMP-s, but also the proportion of MMP present in active form, only capable in vivo of degrading the extracellular matrix, just as the enzymes capable of activate the latent forms. The results were therefore presented in the form of active form/inactive form relationship of the enzyme and the percentages in relation to the proof were calculated to show the effectiveness of the fucose and of the Mixture-1 on its capacity of decreasing the active proportion of the MMP and possibly to inhibit the activation of the inactive form.

TABLE 4
MMP activity of non-stimulated skin explants:
Active form/inactive form relationship of the enzyme
% of effectiveness of the treatments: reduction
of this relationship

| | Active/Inactive | % | Active/Inactive | % of |
|---|---|---|---|---|
| | MMP-9 | effectiveness | MMP-2 | effectiveness |
| Control | 5.48 | — | 0.85 | — |
| Fucose | 2.38 | 57% | 0.80 | 6% |
| 10 μg/ml | | | | |
| Mixture-1 | 3.09 | 46% | 0.67 | 21% |
| 10 μg/ml | | | | |

These results were equally presented in the form of histogram in FIG. 1.

Fucose and the Mixture-1 are capable of decreasing the active form/inactive form relationship of the enzyme. This phenomenon is more important for the MMP-9 than for the MMP-2. The decrease of this relationship implies that the amount of active enzyme, therefore capable of degrading in vivo the extracellular matrix, is less important when the skin explants are cultivated in the presence of fucose or the Mixture-1.

TABLE 5
MMP activity of skin explants stimulated with 1 mg/ml of
hyaluronane
Active form/inactive form relationship of the enzyme
% of effectiveness of the treatments: reduction
of this relationship

| | Active/Inactive | % | Active/Inactive | % |
|---|---|---|---|---|
| | MMP-9 | effectiveness | MMP-2 | effectiveness |
| Control | 7.08 | — | 1.58 | — |
| Fucose | 3.54 | 50% | 1.65 | Ns |
| 10 μg/ml | | | | |
| Mixture-1 | 1.98 | 72% | 0.98 | 38% |
| 10 μg/ml | | | | |

These results were equally presented in the form of histogram in FIG. 2.

The presence of hyaluronane in the culture medium of skin ex10 plants causes an increase in the active form of the MMP-s. Fucose and the Mixture-1 reduce this increase and are, therefore, capable of inhibiting this stimulation of release of active enzymes.

b.2) MMP Activity of the Fibroblasts

When the fibroblasts are cultivated in monolayer, only the widely secreted MMP-2 is visible with a very low quantity of MMP-9, however, present. Only the inactive form of the MMP-2 enzyme is released.

TABLE 6
MMP activity of non-stimulated skin explants
% of effectiveness of the treatments: reduction
of the expression of MMP-2

| | % of effectiveness |
|---|---|
| Control | — |
| Fucose 10 μg/ml | 6% |
| Mixture-1 10 μg/ml | 22% |

These results were equally presented in the form of the histogram of FIG. 3.

One can observe that fucose and the Mixture-1 decrease the expression of MMP-2. The Mixture-1 seems to be more effective than fucose for decreasing this expression.

TABLE 7
MMP activity of skin explants not stimulated with 1 mg/ml
of hyaluronane
% of effectiveness of the treatments: reduction of the
expression of MMP-2 with respect to the control

| | % effectiveness |
|---|---|
| Control | — |
| Fucose 10 μg/ml | 11% |
| Mixture-1 10 μg/ml | 30% |

c) Conclusion

It follows from these experiments that fucose and the Mixture-1 are capable of decreasing the synthesis, as well as the activation of the MMP-2 and MMP-9 by the fibroblasts of human skin. In the same way, fucose and the Mixture-1 are capable of braking, in a more effective way, the superexpression of the metalloproteases in the presence of hyaluronane. Considering the important role of these metallo-endopeptidases in the degradation of the cutaneous extracellular matrix during the aging, this is a greater anti-aging effect.

### EXAMPLE 4

### Activity of Fucose and of the Mixture-1 on the Fibroblasts of Human Skin

The objective of this study was to analyze the action of the Mixture-1 of example 1 on one of the characteristic parameters of the cellular communication and of the action of preventing aging of the fibroblasts of human skin, namely: stimulus of the cellular proliferation of the fibroblasts of human skin.

a) Methodology: Study of the Cellular Proliferation

The fibroblasts of human skin used in this study come from the removal of skin from a woman of 20 years old (26^{th} passing). The cells were cultivated on 12-well plates, in a DMEM culture medium with 10% of fetal calf serum (SVF), 1% of antibiotics and of antifungus (PSF), and 1 μCi/ml of [³H]-timidine (ICN) for 72 hours in the presence of the products to be tested with 2 final concentrations for each sample: 1 μg/ml and 10 μg/ml.

After 72 hours of culture in stove (5% (v/v) CO₂, 95% (v/v) air) at 37° C. in the presence of samples, the cells were washed four times with PBS, then the cellular carpet was detached for 0.05% of tripsin. Three ml of scintillation liquid were then added per sample, then the radioactivity incorporated in the cells is read in a computer with scintillation.

b) Results: Action on the Cellular Proliferation

72-hour incubation of the fibroblasts with the two samples tested (concentrations of 1 μg/ml and 10 μg/ml) significantly stimulates the cellular proliferation in comparison with non-treated cells (see Table 8 below).

TABLE 8
effects of different concentrations of the Mixture-1 on the
cellular proliferation of the fibroblast of human skin
(proliferation with respect to the control)

| | | | |
|---|---|---|---|
| | | Effectiveness with | P with respect to |
| Product | Concentration | respect to the control | the control |

Control

| | | | |
|---|---|---|---|
| Mixture-1 | 1 μg/ml | +48.2% | *** |
| | | | 0.000 |
| Mixture-1 | 10 μg/ml | +30.8% | *** |
| | | | 0.002 |

### EXAMPLE 5

### Cream Against Aging

| Component | % by weight |
|---|---|
| water | q.s.p. 100% |
| Sodium benzoate | 0.2 |
| Di-sodium EDTA | 0.08 |
| Glycerin | 2.00 |
| Butylene glycol | 4.00 |
| Carbomer ETD 2020 | 0.20 |
| Ceteareth-20 | 1.00 |
| Mineral oil | 3.00 |
| Squalane | 2.00 |
| Octyl palmitate | 6.00 |
| Karité butter (“Shea Butter”) | 2.50 |
| Cetearyl alcohol | 1.00 |
| Rosa AFF Rubiginosa seed | 0.20 |
| oil (“Seed Oil”) | |
| Decyl oleate | 0.50 |
| Octyl methoxycinamate | 5.00 |
| Butyl methoxy- | 0.50 |
| dibenzoylmethane | |
| BHA | 0.01 |
| Cyclomethicone | 5.00 |
| Cyclomethicone & Dimethiconol | 2.00 |
| Dimethicone | 2.00 |
| Fragrance (Crematest Feno) | 0.09 |
| Fragrance (Chemoderm) | 0.09 |
| Triethanolamine | 0.30 |
| 2-bromo-2-nitropropane-1,3- | 0.02 |
| diol | |
| Mixture-1 | 0.50 |

### EXAMPLE 6

### Cream Against Aging

| Component | % by weight |
|---|---|
| Water | q.s.p. 100% |
| Sodium benzoate | 0.2 |
| Di-sodium EDTA | 0.08 |
| Glycerin | 2.00 |
| Butylene glycol | 4.00 |
| Carbomer ETD 2020 | 0.20 |
| Ceteareth-20 | 1.00 |
| Mineral oil | 3.00 |
| Squalane | 2.00 |
| Octyl palmitate | 6.00 |
| Karité butter (“Shea butter”) | 2.50 |
| Cetearyl alcohol | 1.00 |
| Rosa AFF Rubiginosa seed oil | 0.20 |
| Ethearyl oleate | 0.50 |
| BHA | 0.01 |
| Cyclomethicone | 5.00 |
| Cyclomethicone & Dimethiconol | 2.00 |
| Dimethicone | 2.00 |
| Fragrance (Crematest Feno) | 0.09 |
| Fragrance (Chemoderm) | 0.09 |
| Triethanolamine | 0.30 |
| 2-bromo-2-nitropropane-1,3-diol | 0.02 |
| Mixture-1 | 0.50 |

1. A mixture of non-sulfated fucose-based oligosaccharides, characterized in that it comprises oligosaccharides of less than 13 saccharide units, comprising at least one fucose unit in a non-reducing end position, and that it is capable of being obtained by means of a process that comprises at least one step of degradation of a polysaccharide from a microorganism of the gender Klebsiella pneumoniae subsp. pneumoniae.

2. A mixture of oligosaccharides according to claim 1, characterized by comprising, with respect to the total weight of the mixture, at least 15% by weight of oligosaccharides of less than 13 saccharide units, comprising at least one fucose unit in a non-reducing end position.

3. A mixture of oligosaccharides according to claim 1 or 2, characterized by comprising, with respect to the total weight of the mixture, from 20 to 50% by weight of oligosaccharides of less than 13 saccharide units, comprising at least one fucose unit in a non-reducing end position.

4. A mixture of oligosaccharides according to any one of the preceding claims, characterized by further comprising, with respect to the total weight of the mixture, from 25 to 45% by weight of oligosaccharides having from 13 to 24 saccharide units, comprising at least one fucose unit in a non-reducing end position.

5. A mixture of oligosaccharides according to any one of the preceding claims, characterized by further comprising, with respect to the total weight of the mixture, from 15 to 35% by weight of oligosaccharides of more than 54 saccharide units, comprising at least one fucose unit in a non-reducing end position.

6. A mixture of oligosaccharides according to any one of the preceding claims, characterized in that the oligosaccharides comprise, at least in part, the motif of recovery fucose-galactose-galacturonic acid.

7. A mixture of oligosaccharides according to any one of the preceding claims, characterized in that it can be obtained by the process that comprises the steps of:
a) causing the microorganism of the gender Klebsiella pneumoniae subsp. pneumoniae to grown in an aqueous nutritive medium by aerobic fermentation of an assimilable source of glucide;
b) recovering the polysaccharide formed from the fermentation must;
c) subjecting the formed polysaccharide to a moderate hydrolysis;
d) subjecting the hydrolysis product of the step c) to an enzymatic hydrolysis; and
e) deactivating the enzyme after recovering the mixture of oligosaccharides thus formed.

8. A mixture of oligosaccharides according to any one of the preceding claims, characterized in that the microorganism Klebsiella pneumoniae subsp.pneumoniae is the microorganism deposited in the Collection Nationale de Cultures de Microorganismes under number I-1507 or a mutant thereof.

9. A mixture of oligosaccharides according to claim 7 or 8, characterized in that the moderate hydrolysis is carried out by a treatment chosen from the group constituted by the treatments with gamma rays, by the protolysis treatments and by the combination of these treatments.

10. A mixture of oligosaccharides according to any one of claims 7 to 9, characterized in that the enzymatic hydrolysis is carried out with at least one endofucosidase.

11. A mixture according to claim 10, characterized in that the endofucosidase is Fermizyme HCP.

12. A cosmetic or pharmaceutical composition characterized by comprising, as a cosmetically or pharmaceutically active agent, at least one mixture of oligosaccharides according to any one of the preceding claims, and at least one cosmetically or pharmaceutically acceptable excipient.

13. A composition according to claim 12, characterized in that it is a dermatological composition.

14. A composition according to claim 13 or 13, characterized in that the mixture of oligosaccharides is present according to a proportion ranging from about 0.001 to about 20% by weight, based on the total weight of the composition.

15. Use of the mixture of oligosaccharides according to any one of claims 1-11, for preparing a composition intended to stimulate cellular communication between the cells of the skin.

16. Use of the mixture of oligosaccharides according to any one of claims 1-11, for preparing a composition intended to stimulate cellular proliferation of the keratinocytes of the skin.

17. Use of the mixture of oligosaccharides according to any one of claims 1-11, for preparing a composition intended to stimulate cellular proliferation of the fibroblasts of the skin.

18. Use of the mixture of oligosaccharides according to any one of claims 1-11, for preparing a composition intended to inhibit the synthesis of the elastase type proteases by the fibroblasts of the skin.

19. Use of the mixture of oligosaccharides according to any one of claims 1-11, for preparing a composition intended to inhibit the superexpression of the proteases MMP-2 and MMP-9 induced by hyaluronane.

20. Use according to claim 18 or 19, characterized in that the composition is intended to inhibit the synthesis of the MMP-2 and MMP-9 proteases by the fibroblasts of the skin.

21. Use according to claim 19 or 20, characterized in that the composition is intended to decrease the sensitivity of the skin to irritation.

22. Use of the mixture of oligosaccharides according to any one of claims 1-11 for preparing a composition intended to stimulate the deposit of collagen fibers on the dermis.

23. Use according to any one of claims 15-22, characterized in that the mixture of oligosaccharides is used according to a proportion ranging from about 0.001 to about 20% by weight, based on the total weight of the composition.

24. Use according to any one of claims 15-23, characterized in that the prepared composition further comprises a cosmetically or pharmaceutically acceptable excipient.

25. A method of cosmetic treatment of the skin, characterized in that one applies to the skin a cosmetic composition comprising at least one mixture of oligosccharides as defined in any one of claims 1-11.

26. A method of cosmetic treatment according to claim 25, characterized in that the cosmetic composition further comprises a cosmetically acceptable excipient.
